(19) United States
(12) Patent Application Publication
(10) Pub. No.: US 2021/0379234 A1
(43) Pub. Date: Dec. 9, 2021

(54) Purified Air Supply System

(71) Applicants: Dong Li, San Ramon, CA (US); Ge Yi, San Ramon, CA (US)

(72) Inventors: Dong Li, San Ramon, CA (US); Ge Yi, San Ramon, CA (US)

(21) Appl. No.: 16/946,093

(22) Filed: Jun. 5, 2020

(51) Int. Cl. A61L 9/20

(52) U.S. Cl. CPC A61L 9/205; A61L 2209/22; A61L 2209/12; A61L 2209/14

(57) ABSTRACT

A air purification system, whose air flow pipeline made of highly reflective and low absorptive material for UVC or UVB light acts also as a UV light waveguide, with a UVC and/or UVB LED built inside the pipeline, is invented. The system is based on the design concept of maximizing UV light exposure dosage to deactivate all the bioaerosols in the air flow pass through the system. The proposed system can be easily integrated into travel pillow, backbag, handbag, belt bag as well as air supply systems for public and private transport systems. The system provides purified air supply for travelers in the closed environment such as in a airplane, or on a train against various dangerous viruses including COVID-19 and SARS virus. It can also be used in office during flu season as well as provide cleaned air supply to its users against the hay-fever.

### FIELD OF INVENTION

The invention is related to air purification system. Particularly, a portable or handhold air supply system to provide sterilized air flow to its users.

### BACKGROUND ART

To keep a human being alive, he or she needs take enough energy from food by eating, and also obtain enough oxygen and get rid of carbon dioxide by breathing. A person may keep himself/herself alive without taking food for a few days but can not survive more than a few minutes without breathing. Just as eating a heathy food, taking in “heathy” air is extremely important to our life quality.

Unfortunately, air can carry quite a lot of pollutants, namely bioaerosols, such as bacteria, mold, viruses, endospores, and even pollen, which can trigger various of infections, allergy and asthma reactions. Some of them can even ultimately lead to complicated short term or long term heath issues. For example, flu is among the top causes of death every year, particularly for the elderly, let alone the recent COVID-19 outbreak.

While the scientists and engineers have been taking a lot of effort with vast capital investment to develop advanced medicine to cure the people affected by contaminated air or to develop vaccine against virus carried by the polluted air, it may be a more efficient approach to develop a portable air purification system to provide people with a heathy air supply, as it is well known that the vaccine against one kind of flu virus may not work well when the virus mutation happens.

Several approaches have been used to provide affordable solution for healthier air supply to ordinary people. It can be classified as physical filtering such as using various air filter; chemically cleaning via oxidization such as Ozone or Photoelectrochemical oxidication (PECO); physical methods such as high temperature disinfection or UV disinfection.

Germicidal UVC (or UV-C with wavelength 100-280 nm) and UVB (or UV-B with wavelength 280-315 nm) can be used for air cleaning. The UVC and low wavelength UVB can make damage on protein in virus and prohibit its reproduction activity. UVC and UVB light can even efficiently inactivate organic bioaerosols such as multi-drug-resistant bacteria, differing strains of viruses. The basic theory behind this application is that the UVC and low wavelength UVB can deactivate pathogenic bacteria, viruses and other microorganisms via formation of thymine dimers in deoxyribonucleic acid (DNA) or ribonucleic acid (RNDA), which prevents further replication of the DNA or RNA strain. It is worth to note that the maximum absorption wavelength of DNA or RNA is approximately 260 nm, therefore UVC is much more efficient than UVB.

The widespread use of germicidal ultraviolet light in public settings has been very limited because UV light, particularly UVB, UVA and high wavelength UVC light, are a human health hazard, being both carcinogenic and cataractogenic. Secondly, the conventional UVC sources, which are the most efficient one for germicidal purpose, are Low- or medium-pressure mercury vapor lamps with a high operating voltage on the order of 1-10 kV, and a high-power UV radiation (on the order of 10 W) at a wavelength of 254 nm—close to 260 nm, which are not for the portable, particularly for handhold devices. There are many drawbacks to using mercury vapor lamps; for example, the lamps contain highly toxic mercury sealed in a fragile quartz glass tubes, which is easy to break and contaminate the environment. The lamps have a long warmup times of approximately 10 min.

UVC, light-emitting diodes (DUV-LEDs, UVC LEDs), a solid light source based on carrier injection into multiquantum well (MQW) semiconductor layer, has numerous advantages and may provide solutions to the above drawbacks of UV mercury lamps for portable and handhold air cleaning devices. The issue of existing DUV LED is its really low external quantum efficiency (only 1% to a few % for the time being), which means that, to achieve high output power, a significant input power needed with majority of power turning into heat. This demands a solution for quick heat dissipation.

It is not easy to make a UVC LED working on a handhold air purification device. One one hand, considering UVC LED's low output efficiency and challenge on heat dissipation for keeping the device alive, only UVC or UVB LED with output power of a few mW to tens of mW can be used on the portable or handhold system. On the other hand, to make air cleaning work, the bioaerosols need to expose under enough UVC or UVB dosage (or area density dosage) or enough accumulated light energy to trigger the dimmer formation. This puts forward a great challenges on system designers to answer the question—how to use the lower output UVC or UVB LED to provide enough energy exposure to terminate the DNA's reproduction in the incoming bioaerosols within the air stream.

The invention proposed here provides a solution for this dilemma for portable or handhold air cleaning device based on UVC and/or UVB LED.

### SUMMARY OF THE INVENTION

In this invention, we propose a novel design of air cleaning device based on the UVC and/or UVB LED.

The concept of this invention is to make the system's air flow pipeline act also as UVC or UVB light waveguide by using the high UVC and/or UVB reflection (>75%) and low UVC and/or UVB absorption material at least on the internal surface of air flow pipeline. By doing so, it provides enough UVC and/or UVB light exposure to deactivate all the incoming bioaerosols in the supplied air, therefore offers a cleaned air stream to its user.

The current design also provides a novel design for UVC or UVB LED heat dissipation by placing the LED inside the air flow pipeline, which enables air cooling happening when the air flow passes by. Moreover, the heat exchange between the air flow and LED can increase the air stream temperature slightly above the ambient air temperature. By doing so, the air stream out from the system can expel the ambient air away from its user(s) to enable its user(s) only breath the purified air out from the system.

This design allows everything in the air flow through the system, from the air inlet to the air outlet, to experience as much UV exposure as possible. The instant-on capability of UVC LED or UVB LED together with extra fast light speed compared with slow air flow speed enable air purification starts immediately after the system is powered on.

Moreover, at least one component coated by metal oxide nanoparticles such as TiO2, or ZnO together with or without electrically-isolated metal nano-particles (NP), are added into the proposed system firstly to further enhance the cleaning functionality and secondly to avoid UV light escaping from the system by promoting UV light absorption. The incoming UV light will induce plasmonic resonance in the metal nano particles, to either generate the plasmon induced heating or re-emit locally enhanced UV light around the NP at the interface between the NP and air. Several materials, such as Al, Ga, Rh will be used for such purpose.

Considering the UV light interacting with oxygen in the air could potentially generate some small amount of ozone, the system also implements an activated carbon filter near the air outlet inside the air flow pipeline purely for the purpose of removing the small amount of ozone from air supply to its user.

To provide cooling to the UVC and/or UVB LED, we implement an optional thermal electrical cooler (TEC) to accelerate the cooling for LED and also to warm up the output air above the ambient temperature, which provides an expelling force to the ambient air as well.

The proposed air purification system provides a solution to good balance between air flow, UV light propagation, heat dissipation and power consumption. It is very useful for travelers in the closed environment such as in an airplane, or on a train. It can also use in office during flu season particularity for the elderly as well as provide cleaned air for users against the hay-fever. It is also a cheap tool against coronavirus such as COVID-19 or SARS.

### BRIEF DESCRIPTION OF THE DRAWINGS

FIG. 1 a schematic embodiment of the system proposed in this invention to use highly UV reflective material to form the air flow pipeline acting also as waveguide for UV light together with the UVC or UVB LED inside the air flow pipeline for better cooling.

FIG. 2 a schematic embodiment of system passive (without need of power) and active (need power) components for the proposed system by following air path through the system along with a power management subsystem.

FIG. 3 an embodiment of a component coated with Nano particles of metal oxide mixed with electrically isolated nanoparticles of Aluminum (Al), Gallium (Ga) or previous metal for plasma enhanced UV light absorption and locally enhanced UV cleaning.

FIG. 4 an embodiment of the proposed system integrated inside a travel pillow.

FIG. 5 an embodiment of the proposed system integrated inside a backpack.

FIG. 6 an embodiment of the proposed system integrated inside air supply system on a plane.

### DETAILED DESCRIPTION

The following numerous specific detail descriptions are set forth to provide a thorough understanding of various embodiments of the present disclosure. It will be apparent to one skilled in the art, however, these specific details need not be employed to practice various embodiments of the present disclosure. In other instances, well known components or methods have not been described.

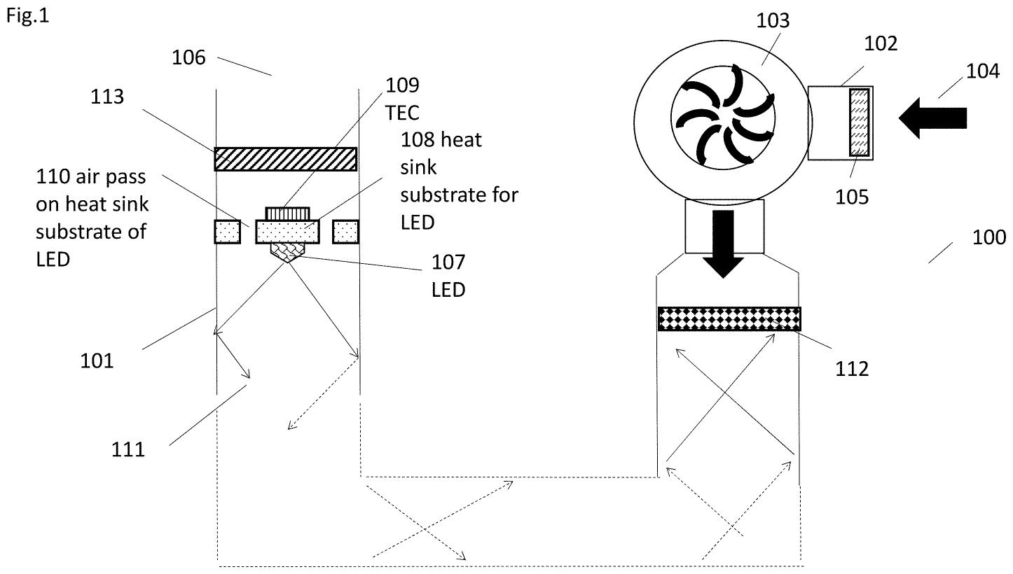

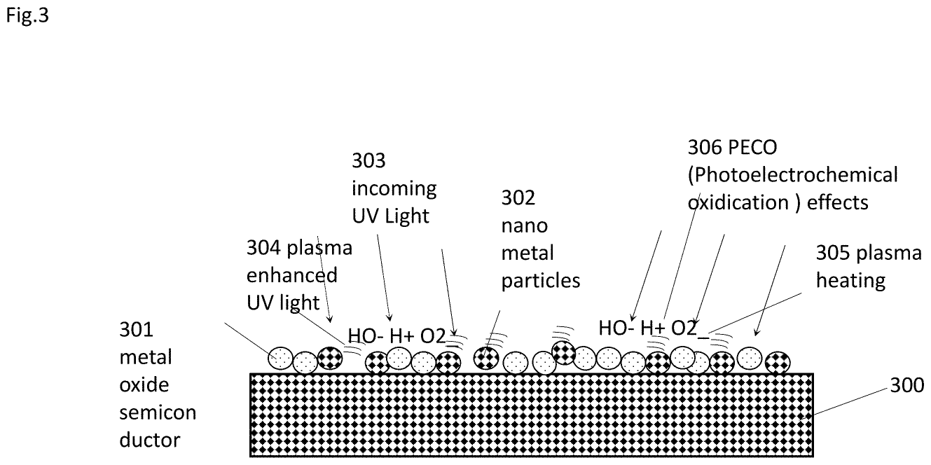

FIG. 1 a schematic embodiment of the system proposed in this invention to use highly UV reflective material to form the air flow pipeline acting also as waveguide for UV light together with the UVC or UVB LED inside the air flow pipeline for better cooling. As mentioned previously, the design principle of the proposed system is to allow everything in the air flow to experience maximum UV exposure from the air inlet to air outlet by using the air flow pipeline as a UVC or UVB light waveguide based on the reflection from the internal surface of the air flow pipeline made of UVC/B highly reflective and low absorption material(s). By doing so, it can leverage low power UVC or UVB LED for air purification purpose, particularly for portable device or equipment with small footprint. For the purpose of simplicity, in FIG. 1, only the internal surface of the air pipeline is shown and represented here. Having said that, the highly UV light reflective material for internal surface of the air flow pipeline and the body of the air flow pipeline can certainly the same materials such as Polytetrafluoroethylene (PTFE). As shown in the figure, the purified air supply system 100 has more or less its whole air flow pipeline 101 or at least its internal surface made of highly UV light reflective material with low UVC or UVB absorptions. The air flow pipeline 101 has air inlet 102, near which a air intake device 103 eg. A fan is used to bring air 104 from ambient air into the system. The simple air filter 105 is used near air inlet 102 to stop the dust and large particles getting into the system so that air flow through the system from air inlet 102 to air outlet 106 without much blockage. Insider the air pipeline 101, a UVC or UVB LED 107 is mounted on its mounting substrate 108, which acts also as heat sink for the LED 107 and has air passing hole 110 in it. With the air passing hole 110 on the mount substrate 108, the smoothness of air steam is not blocked by the introduction of the LED 107 and its mounting substrate 108 in the pipeline 101. The air flow 104 taken into the system also can cool the LED 107 via heat exchange between air flow and LED 107/its mounting substrate 108. While the LED 107 gets cooled down, the air gets warmed up, which allow the air steam out from air outlet 106 having a slightly higher temperature than the ambient air. This allows the air flow/stream out from the outlet 106 to expel the ambient air to provide the user(s) of the system with purified air only. A optional TEC (thermoelectric cooling device) 109 can be mounted on substrate 108 to provide an accelerated cooling for LED 107 depending on the choice of the power and efficiency of UVC/UVB LED 107. The UV light 111, indicated by arrows both solid or dotlined, emitted from the LED 107 is reflected by the highly reflected and low UV absorption internal surface of air pipeline 101 along it path, from one end to the other end of the air flow pipeline, and eventually arrived at 112—an air passing UV light absorption component coated with nano particles of metal oxide semiconductor with or without metal nanoparticles. The detailed of air passing component 112 will be illustrated later in FIG. 3. It is worthwhile to note that the arrangement and exact location of LED 107 along with its associated components 108 and 109 as well as light absorption 112 can be varied along the air flow pipeline 101 because the light propagation speed is very fast. Therefore there are a lot of freedom to adopt different arrangement for LED related optical and mechanical components other than what is being shown here. The current embodiment is only used to illustrate the principle of the proposed system design. There is also an optional activated carbon filter 113 near the air outlet 106 to eliminated any possible of a small amount of ozone generated by UV light when it interacted with oxygen molecules in the air flow.

For any bioaerosols in the air flow 104 into the system, it has to follow the air flow pipeline from air inlet 102 to air outlet 106, during which it has a lot of chance to interact with continue UV light emitted from LED 107. Provided there is enough dosage under the UV light exposure, the bioaerosols will be killed or deactivated by the UV light exposure. In other words, the UV light literally acts as light sword to slaught the bioaerosols and help to purified the air flow thus provides its user an air stream which has been already cleaned or decontaminated by the proposed system.

There are quite a few choice of materials with high reflection and low absorption for UVC/B light. They are general PTFE film or tube (eg. those from Gore); or ePTFE (expanded PTFE) film or tube; or porous PTFE film or tube; film of Nitrocellose; or even Nitrocellose pain with special components (without those for high UVC/B absorption); Teflon tape/film and tube; Aluminum foil or tube; Tetratex film or tube from Tatratec Corp; 3M's enhanced spec reflector (ESR) film/sheet; Dupont's Tyvek paper or Melinex film/sheet; or Toray's Lumirror sheet; or PVDF maded sheet or tube.

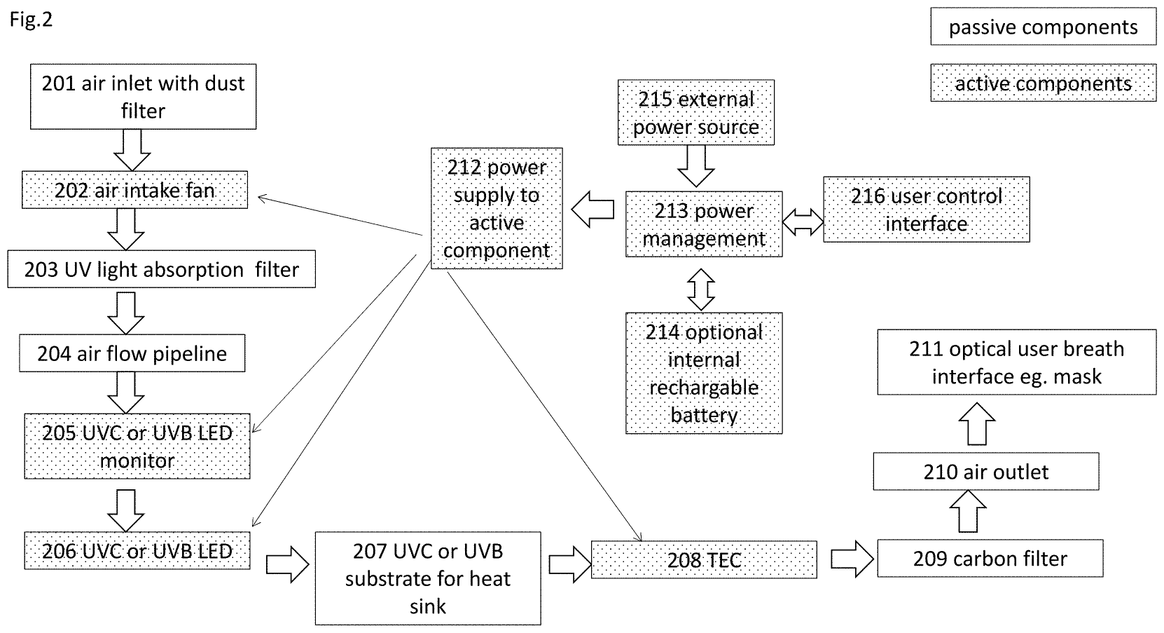

FIG. 2 shows a schematic embodiment of system passive (without need of power) and active (need power) components for the proposed system by following air path through the system along with a power management subsystem. While the schematic embodiment shows like a flow chart, it is worthwhile to note that the exact location of the components can vary as long as the function of the whole system is intact. As shown, the passive and active components are differentiated via without or with patterned text frames. Ambient air was taken into the system via air intake fan 202 at air inlet with a dust filter 201. Large particles and dust in the air will be removed at the dust filter 201. Air then arrives at UV light absorption filter 203, where it get cleaned as a few processes, such as plasmonic cleaning, local UV enhanced, local heating and PECO effects, happen simultaneously. The processes at 203 can enhance each other while the UV light gets absorbed by various physical processes. Once the air enters into air flow pipeline 204, which is made or whose internal surface is made by highly UV reflective and less absorption material(s). With the continuous UV light emitted from UVC or UVB LED 206, whose heath is monitored by its monitor 205, being reflected and its intensity being enhanced inside the air flow pipeline 204, any bioaerosols in the air flow will experience multiple chances to hit by the UV light, which will terminate its bioactivity. Effectively, the harmful bioaerosols inside the air steam will be killed, which allows air flow gets purified. The LED 206 is mounted in its mounting substrate 207, which also acts as its heat sink. One optional TEC 208 can be introduced to further enhance the cooling of the LED 206. Before air exits the system at air outlet 210, it needs pass a carbon filter 209 to filter away any trace of ozone generated by the system and also remove any odor if there is any existing in the air flow to provide the system user purified air with fresh smell. A optional user breath interface such as a facemask can be provided as an accessory of the system. A group of electronic components are needed to provide necessary power for the active components of the system. They are power supply to active components 212 including voltage regulator for example; a system power management chip 213, which can take electrical power from either external power source 215 such as main power plug or a USB connector, or a optional internal rechargeable battery 214; together with a user control interface with power on button, other control buttons, and system heath indicators for battery capacitance, and/or UV LED heath and lifttime etc.

FIG. 3 shows an embodiment of a component coated with Nano particles of metal oxide mixed with electrically isolated nanoparticles of Aluminum, Gallium (Ga) or other previous metal for plasma enhanced UV light absorption and locally enhanced UV cleaning. This represents as the component 112 in FIGS. 1 and 203 in FIG. 2. As shown, the component 300 is coated with mixture of nanoparticles, which includes metal oxide 301 and precious metal 302. The metal oxide 301 can be either photocatalyst NPs and/or metal oxide semiconductor, such as Titanium dioxide (TiO2), or Zirconium oxide (ZrO), or Zinc oxide (ZnO), or Magnesium oxide (MgO), or tungsten trioxide (WO3), or the combinations of the above mentioned materials, while the electrically isolated nanoparticles can be either Aluminum (Al), Gallium (Ga) or precious metal nanoparticles such as Pt, Au, Ru, Rd, Rh. The component 300 can be made by UV highly reflective and low UVC/B absorptive solid film or porous film.

When the incoming UV light, indicated here by arrow 303, reaches the surface, there are a few processes happening at the same time. Firstly at the surface of metal oxide nanoparticles, there is a UVC/B induced process 306, which can be either photocatalyst effect (if the incoming air is dry) or PECO effects (if the incoming air has high moisture) which can assist the air purification for the system. It is worth to note that our proposal here is different from the normal PECO systems in the market, which uses UVA light and also the catalyst particles is deposited on air filter(s). Here, the proposal catalyst is deposited on high UVC/B reflective and low UVC/B absorptive substrate to enhance the interaction between the photons and NPs of photocatalyst. Also the proposed system expect to work well for dry air with low humidity based on photocatalyst effect alone. Secondly, there is plasmonic effects either plasma enhanced UV intensity local increase 304 (plasmonic light enhancement effect) and plasma heating effect 305 (plasmonic photothermal effect) happening at the interface between air and electrically isolated nano metal particles. Both effects can help air cleaning as well. For material used for the plasmonic device, isolated nano metal particles in the size range from 5 nm-100 nm made from Aluminium (Al) with AlOx, Ga with its native oxide, even more expensive Rh, or their combinations as either an alloy system or an composite system can be used.

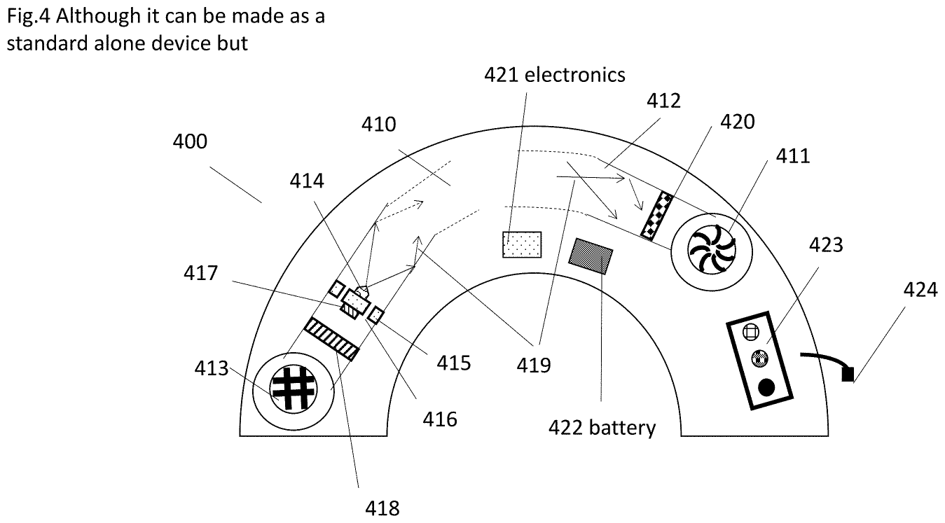

FIG. 4 shows an embodiment of the proposed system integrated inside a travel pillow. The travel pillow 400 with a built-in purified air supply system 410 is shown here. The system 410 comprises air inlet with a fan 411, which takes the ambient air into the air flow pipeline 412. The air exits the system 410 at air outlet 413. The pipeline 412 or at least its internal surface is made of high UV reflective and less absorption material. Inside a pipeline there is a UVC or UVB LED 414 which injects UV light into the pipeline 412. The LED 414 is mounted on its substrate 415. which has air passing hole 416 to allow air passing smoothly without being impacted much by the installation of the LED 414 and its mounting substrate 415. An optional TEC 417 can be introduced to further enhance the cooling of the LED 414. The light indicated here by arrow 419 emitted from LED 414 travels along the pipeline and almost instantly saturates the space inside the pipeline since LED 414 is turned on due to multiple internal reflections from the material of pipeline and ultrafast light speed in air. Whenever the light 419 meets any bioaerosols, with enough light density and/or accumulated energy, it will terminate the bioactivity of the bioaerosols thus purify the air passing through the system. It is worthwhile to mention that an optional carbon filter 418 can be used near the air outlet 413 to remove any trace of ozone or odor from the air flow. The extra UV light will be absorbed at the component 420 coated with various nanoparticles, whose details have been given in FIG. 3. To support the active components of the system, electronics 421 and rechargeable battery 422 are introduced into the system together with control interface 423 for the system user. An external charging cable 424 is also included on the travel pillow to provide charging capability for battery 422. The travel pillow with the proposed system provides a good choice for people travelling on plane and train during flu seasons or whenever an epidemic is ongoing.

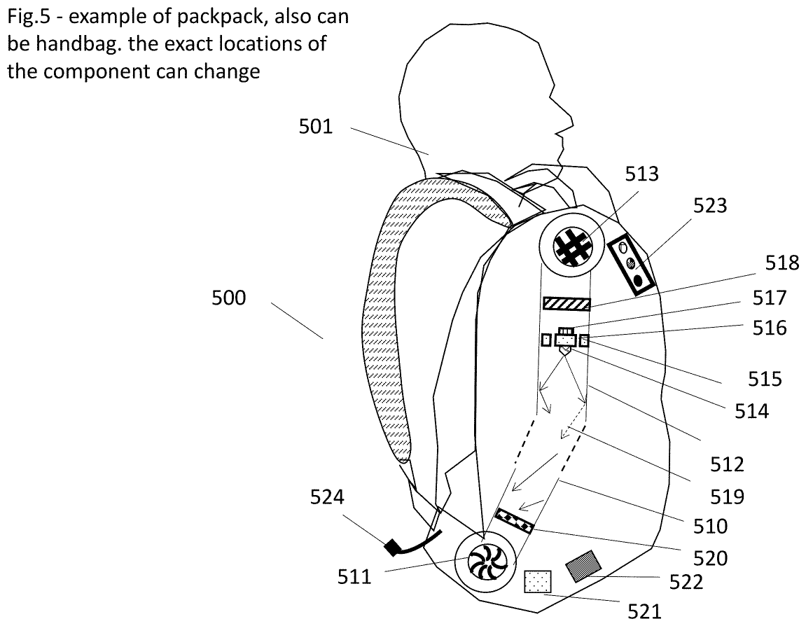

FIG. 5 shows an embodiment of the proposed system integrated inside a backpack. Although an example of backpack is shown here, similar concept can be adopted for belt bag, handbag and other similar personal items. The backbag 500 integrated with proposed system 510 provides purified air to its user 501. The system 510 comprises air inlet with a fan 511, which takes the ambient air into the air flow pipeline 512. The air exits system at air outlet 513. The pipeline 512 or at least its internal surface is made of high UV reflective and less absorption material. Inside a pipeline there is a UVC or UVB LED 514 which injects UV light into the pipeline 512. The LED 514 is mounted on its substrate 515. which has air passing hole 516 to allow air passing smoothly without being impacted much by the installation of the LED 514 and its mounting substrate 515. An optional TEC 517 can be introduced to further enhance the cooling of the LED 514. The light indicated here by arrow 519 emitted from LED 514 travels along the pipeline and almost instantly saturates the space inside the pipeline since LED 514 is turned on due to multiple internal reflections from the material of pipeline and ultrafast light speed in air. Whenever the light 519 meets any bioaerosols, with enough light density and/or accumulated energy, it will terminate the bioactivity of the bioaerosols thus purify the air passing through the system. It is worthwhile to mention that an optional carbon filter 518 can be used near the air outlet 513 to remove any trace of ozone or odor from the air flow. The extra UV light will be absorbed at the component 520 coated with various nanoparticles, whose details have been given in FIG. 3. To support the active components of the system, electronics 521 and rechargeable battery 522 are introduced together with a control interface 523 for the system user. An external charging cable 524 is also included to provide charging capability for battery 522. The backbag with the proposed system provides a good choice for people travelling on public transport systems such as tram, train, bus, van, plane during flu season or whenever an epidemic is ongoing.

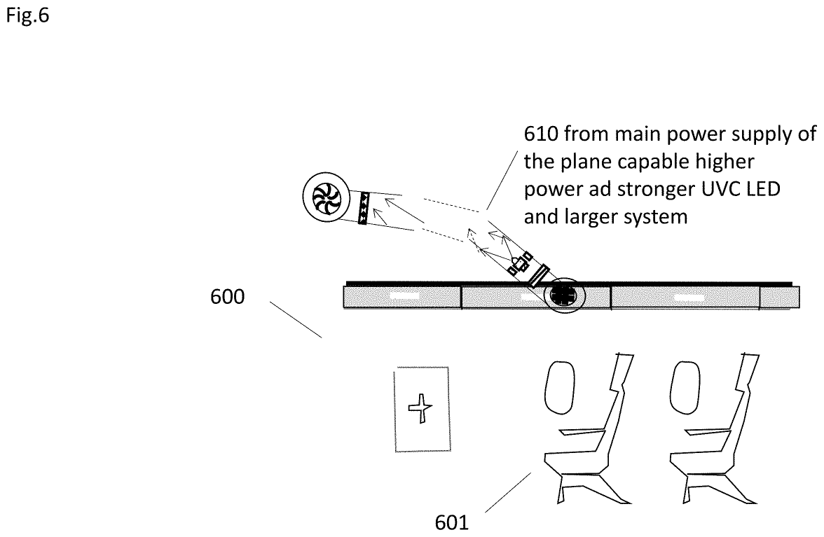

FIG. 6 shows an embodiment of the proposed system integrated inside air supply system on a plane. For the reason of simplicity, there is no need to repeat every details again. As we all know, there is a air supply exit above every seat on a plane, The plane also has a oxygen supply system for every seat. The idea proposed here is to integrate the proposed system into either of these two systems. In FIG. 6, the schematic drawing illustrates the idea of this system 610 is built as part of the air supply system 600 to supply air to a customer sitting on the seat 601. The advantage of the proposal is that this provides purified air supply to every customer on the plane and prevent the bioaerosols particularly any virus spreading on broad to affect large population on plane. Similar ideas can be implemented for other kinds of public and private transport systems.

What is claimed is:

1. An air purification system comprises at least:
An air flow pipeline, with an air inlet and an air outlet, which carries an air flow taken from ambient air, is made of or its internal surface is made of a highly reflective material with low light absorption for light of an ultraviolet of c-band (UVC) and/or an ultraviolet b-band (UVB) band;
A ultraviolet c-band light emitting diode (a UVC LED) or a ultraviolet b-band light emitting diode (a UVB LED) inside the air flow pipeline as a ultraviolet light source emitting a batch of light into the air flow pipeline, inside which the air flow is cleaned via a ultraviolet light exposure.

2. The system of claim 1, wherein said UVC LED or said UVC LED is cooled by said air flow through a thermal exchange process when said air flow is passing by the UVC LED or the UVB LED.

3. The system of claim 2, wherein said thermal exchange process heats up the bypassing air flow to a predetermined temperature above ambient air temperature.

4. The system of claim 1, wherein said UVC LED or said UVB LED has a power density above a predetermined value to ensure a group of concerned airborne micro-organism and/or a group of concerned bioaerosols are killed by said ultraviolet light exposure.

5. The system of claim 4, wherein said group of concerned airborne and/or said group of bioaerosols include a group of bacteria, molds, viruses, endospores, and pollens.

6. The system of claim 5, wherein said group of viruses includes COVID-19 virus, SARS virus, and their variations.

7. The system of claim 1, wherein said air purification system further comprises a device to monitor the heathy status of said UVC LED or said UVB LED and to report the malfunction of said UVC LED or said UVB LED in time for a maintenance of the system.

8. The system of claim 1, wherein said air purification system further comprises an electrical power subsystem, which provides power to the system via either external power source, or at least an internal rechargeable battery, or both.

9. The system of claim 1, wherein said UVC LED or said UVB LED is mounted on a metal substrate, which acts as a heat sink for a purpose of heat dissipation to cool down said UVC LED or said UVB LED.

10. The system of claim 3, wherein said predetermined temperature above ambient air allows the air flow coming out from the system to have an expel force to a batch of untreated surrounding ambient air to ensure a user of the system only breath a stream of purified air coming out of the system.

11. The system of claim 9, wherein said heat sink has an attached thermoelectric cooler (a TEC) mounted on it to enhance cooling for said UVC LED or said UVB LED to ensure its working temperature within a predetermined safe range.

12. The system of claim 11, wherein said TEC is inside the air flow pipeline to enable a thermal exchange it and bypassing air flow.

13. The system of claim 1, wherein said air flow pipeline comprises at least an activated carbon filter near said air outlet to remove a small amount of ozone in said air flow before reaching to said air outlet.

14. The system of claim 1, wherein said highly reflective material with low light absorption is either a piece of PTFE film and/or a PTFE tube; or a piece of ePTFE (expanded PTFE) film or a ePTFE tube; or a piece of porous PTFE film or a porous tube; or a piece of Nitrocellose film; or a kind of low UV absorption Nitrocellose paint; or a piece of Teflon tape/film and/or a Teflon tube; or a piece of Aluminum foil and/or a Aluminum tube; or a piece of Tetratex film and/or a Tetratex tube from Tatratec Corp; or a piece of 3M's enhanced spec reflector (ESR) film/sheet; or a piece of Dupont's Tyvek paper; or a piece of Dupont's Melinex film/sheet; or a piece of Toray's Lumirror sheet; or a PVDF tube or a piece of PVDF sheet.

15. The system of claim 1, wherein said air purification system further comprises at least a component coated with at least a layer of nanoparticles of either Titanium dioxide (TiO2), or Zirconium oxide (ZrO), or Zinc oxide (ZnO), or Magnesium oxide (MgO), or tungsten trioxide (WO3), or the combinations of the above mentioned photocatalyst with or without an addition of a small amount of electrically isolated nano particles of Aluminum (Al), or Gallium (Ga), or a precious metal to absorb a batch of light outcome near said air inlet and/or outlet of said air flow pipeline.

16. The system of claim 1, wherein said air purification system is integrated as a part of either a air supply system or a oxygen supply system for a transport system.

17. The system of claim 16, wherein said transport system is either a plane, or a train, or a bus, or ferry, or van, or a tram, or a car.

18. The system of claim 1, wherein said air purification system is integrated as a functional part of either a travel pillow, or a backpack, or a handbag, or belt bag.

19. The system of claim 1, wherein said air purification system further comprises a air filter near said air inlet to block a group of dust and particles in the ambient air.

20. The system claim 1, wherein said air purification system further comprises a power subsystem including a group of predetermined electronics and at least a rechargeable battery.
